(19) United States
(12) Patent Application Publication
(10) Pub. No.: US 2008/0097472 A1
(43) Pub. Date: Apr. 24, 2008

(54) EPISIOTOMY AID DEVICE

(76) Inventors: Jonathan AGMON, Ra'anana (IL); Arnon AGMON, Tel Aviv (IL)

Correspondence Address: SOROKER-AGMON ADVOCATE AND PATENT ATTORNEYS, NOLTON HOUSE, 14 SHENKAR STREET, HERZELIYA PITUACH, 46725, omitted

(21) Appl. No.: 11/874,252

(22) Filed: Oct. 18, 2007

(30) Foreign Application Priority Data
Oct. 22, 2006 (IL) IL 178801

(51) Int. Cl. A61B 17/42; A61B 17/03

(52) U.S. Cl. 606/119; 606/215

(57) ABSTRACT

A device disclosed is a device for protecting soft tissues and for assisting in the performance of surgical procedure involving incision of tissues. The incision having a longitude section and two ends, and the device comprises a flexible sheet configured to be placed on the tissue, wherein the sheet outlines the tissue. According to one preferred embodiment the device provides indication for the location for performing the incision. According to further embodiments the device can comprise attaching means for attaching the device.

### FIELD OF THE INVENTION

The present invention relates generally to a device for assisting with the performance of a surgical incision, and particularly to a device assisting within a performance of episiotomy.

### BACKGROUND OF THE INVENTION

Episiotomy is a prophylactic incision performed at the perineum of a woman delivering a new born baby. Episiotomy is performed in order to facilitate a birth of an infant and to prevent further tear of the vaginal wall and perineum. It is performed during the end of the second stage of delivery, immediately before the head delivers by simple incision to the perineum and part of the vaginal wall combined (referred hereafter for practical use as the fourchette). Episiotomy is also performed during any instrumental delivery such as vacuum extraction or forceps delivery in order to facilitate such deliveries and provide more space for handling devices used in such deliveries. An important consideration for performing episiotomy in all cases is to reduce the risk of tears to a minimum. Thus, episiotomy is an extremely common practice in obstetrics.

The use of the episiotomy for prevention of further vaginal tear has been questioned. Some studies showed no advantages are reached during delivery when initiating an episiotomy. However, many obstetricians and midwifes feel it can facilitate the last minutes of the second stage of labor and reduce the extent of perineal and vaginal tears, especially in nulliparas. During difficult labor the first maneuver to be performed is an episiotomy with the intention of reducing the pressure on maternal perineal and vaginal tissue and to allow performance of special maneuver to facilitate birth. The previous experience of the midwife or physician delivering women is the most important factor whether episiotomy is performed or not. In many cases the attitude of the medical establishment can deter or encourage a performance of an episiotomy.

Episiotomy can be performed in two fashions; the midline and the mediolateral types. Each has it advantage and disadvantage. In short, the midline incision is more cosmetic and heals better but extension to the anal and rectal tissue is more common while the mediolateral type is safer in the later respect but healing and cosmetic results are in inferior.

While performing an episiotomy the tissue opened includes coetaneous, subcutaneous and in some occasions perineal muscle or external sphincter muscle (third degree tear). A tear which extends to the anal or rectal mucosa (fourth degree) is more complicated to repair and may need the interference of a proctologist surgeon specialist.

One problem with episiotomy is the continuation of the episiotomy into the vagina or in the case of midline episiotomy into the anal mucosa. This may result due to pressure of the delivering parts on already tear tissue allowing the continuation of the cut tissue in its most weak part. The performance of the episiotomy is intended to be preventive for other tears but it is not preventive to the extension of itself. This may complicate closure of the prophylactic severed tissue and may discourage some practitioners from performing such prophylactic incision in the first place.

Yet another problem with episiotomy is the cosmetic results of the repair especially in mediolateral incisions. In such cases the cut tissue may not be easy to approximate due to lack of recognizable clear landmarks and distortion of the tissue. The laxity and elasticity of the perineal and vaginal tissue as well as extended tears of the episiotomy are probably the cause of this problem.

It is the purpose of this invention to provide an instrument of multipurpose that will outline the edges of the prophylactic incision, prevent extension of the incision, allow easy and safe correction of the defect created by the incision and provide within the device itself the means to correct the defect in a simple, safe, cosmetic and fast fashion.

### SUMMARY OF THE INVENTION

An aspect of some embodiments of the invention relates generally to a device for assisting in the performance of surgical procedure involving an incision of a tissue.

An aspect of some embodiments of the invention relates to a device for the performance of episiotomy.

An aspect of some embodiments of the invention relates to a flexible frame configured to be placed on a tissue encircling the incision, said incision having a longitude section and two ends.

An aspect of some embodiments of the invention relates to a deformable flexible frame enabling deformation of the frame relative to the tissue.

An aspect of some embodiments of the invention relates to a frame to be used in episiotomy that can be deformed and placed adjacent to the tissue.

An aspect of some embodiments of the invention relates to a deform able device wherein the deformation advances with the tissue bordering the incision.

An aspect of some embodiments of the invention relates to a device enabling to perform surgical procedure involving an incision in a safe manner, diminishing risk of further unnecessary tearing of the tissue.

An aspect of some embodiments of the invention relates to a device for providing repair assisting means for repairing an incision.

According to one preferred embodiment of the present invention comprises a device for protecting soft tissue and for assisting in the performance of surgical procedure involving incision of tissue, the incision having a longitude section and two ends, the device comprising a flexible sheet configured to be placed on the tissue, wherein the sheet outlines the tissue. The device is flexible and has a frame. The device can be placed outlining the tissue. The device's flexibility enables deformation of the device relative to the tissue. The device comprises, at least two opposing sides; and at least one end, wherein the sides and the end are integrally connected. The device is configures to be placed adjacent to one of the ends of the incision. The device's frame is configured to be placed adjacent to the end of the incision or the longitude section of the incision. The device comprises at least one flexible section enabling the deformation of the frame. The flexible section can comprise a hinge. The device comprising attaching means such as adhesive or glue or pins. The device repair assisting means for assisting repair of the incision.

### BRIEF DESCRIPTION OF THE DRAWINGS

The present invention will be understood and appreciated more fully from the following detailed description taken in conjunction with the drawings. Identical structures, elements or parts, which appear in more than one figure, are generally labeled with a same or similar number in all the figures in which they appear, wherein:

FIG. 1 is a schematic illustration of a device according to a first exemplary embodiment of the present invention, before the performance of episiotomy;

FIG. 1A is a side view of a device according to the first exemplary embodiment placed on a fourchette;

FIG. 2 is a schematic illustration of a device according to the first exemplary embodiment placed on a fourchette shown after performance of episiotomy;

FIG. 3 is a top view of a device in accordance to the first exemplary embodiment;

FIG. 4 is a a side view of a device in accordance to the first exemplary embodiment;

FIG. 5 is a top view of a device according to a second exemplary embodiment according the present invention;

FIG. 6 is a side view of a device according to the embodiment shown in FIG. 5;

FIG. 7 is a bottom view of a device according to the embodiment shown in FIG. 5;

FIG. 8 is a cross section view of the end of a device according to the embodiment shown in FIG. 5;

FIG. 9 is a top view of a device in accordance to a further exemplary embodiment;

FIG. 9A is a side view of a device in accordance with the embodiment shown in FIG. 9.

FIG. 10 is a cross section view of the end of a device according to the embodiment shown in FIG. 9;

FIG. 11 is a top view of another exemplary embodiment of the present invention;

FIG. 12 is a close view of repair assisting means in accordance with the embodiment shown in FIG. 11;

FIG. 13 is a top view of a device according to a further embodiment of the present invention;

FIG. 14 is side view of the embodiment shown in FIG. 14;

FIG. 15 is a view of the embodiment shown in FIG. 13 placed on a fourchette;

FIG. 16 is a view of the embodiment shown in FIG. 13 placed on a fourchette after an incision was made;

FIG. 17 is a view of the embodiment shown in FIG. 13 placed on a fourchette after an incision was made and wherein the device is shown in a top view.

### DETAILED DESCRIPTION OF THE INVENTION

The present invention provides a device for assisting in the performance of surgical procedure involving an incision of a tissue. The device comprises a flexible frame configured to be placed on a tissue encircling the incision. The incision has a longitude section and two ends. The frame's flexibility enables deformation of the frame relative to the tissue. Thus, given the shape of the tissue (e.g. planar, angular, wave, hyperbolic, sinusoidal, chaotic, combination thereof and others) the frame can be deformed and placed adjacent to the tissue. Furthermore, in some embodiments the device deformation advances with the tissue bordering the incision. The device disclosed enables to perform surgical procedure involving an incision (e.g. episiotomy) in a safe way, diminishing risk of further unnecessary tearing of the tissue. Moreover, the device, as will be apparent below, in some preferred embodiment provides repair assisting means for repairing the incision. The term tissue as used throughout the application refers to a single tissue or tissue layer as well as to a part of a tissue, or a number of tissues. Thus, one skilled in the art can easily comprehend the modifications required while referring to the term tissue Persons skilled in the art will also appreciate that the present invention can be applied to treating both animal and humans females. The present invention may be applied to cows, horses, and other animals having large fetuses.

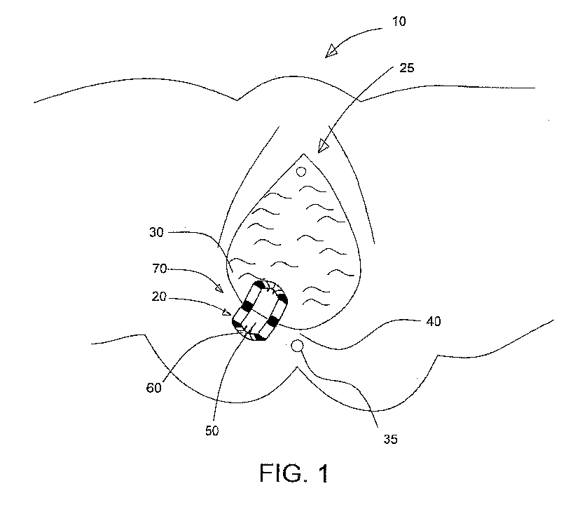

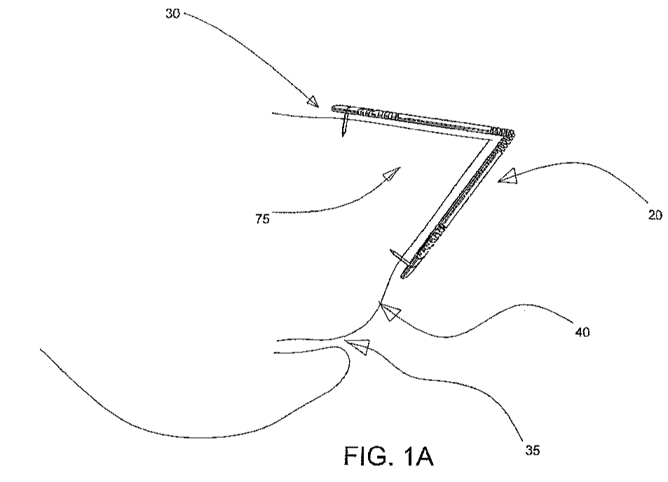

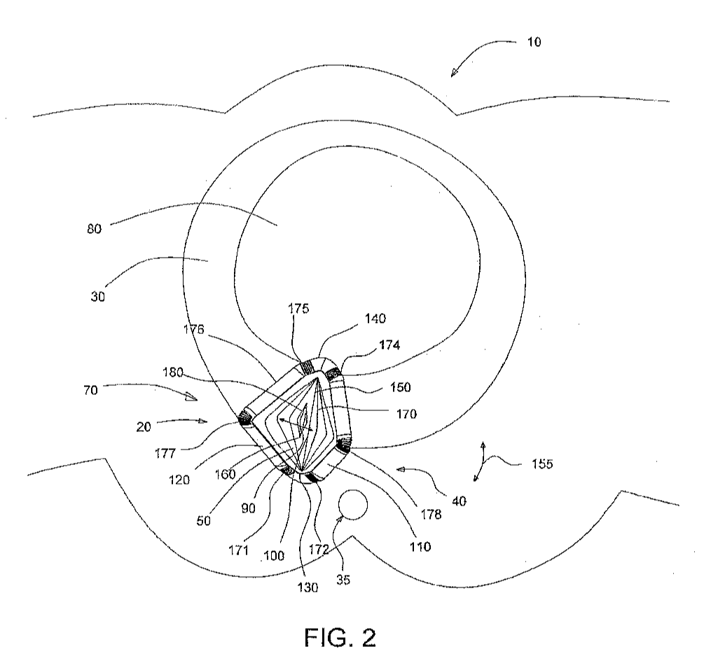

Referring to the drawings, FIG. 1 is an illustration of a perineum of external genitalia of a female 10, showing anus 35 perineum 40 and vagina 25. An episiotomy aid device of the invention, generally designated 20, is placed partially on the vagina wall 30 and partially on the perineum 40, hereafter referred to as fourchete tissue 70. FIG. 1 is a schematic illustration of a device 20 according to a first exemplary embodiment of the present invention. Device 20 is placed on a fourchette 70 before performance of episiotomy. Device 20 can be placed on fourchette 70 of delivering female 10 at any given time, prior to, or during the end of the second stage of delivery, immediately before the infant head delivers. Device 20 outlines the area where the episiotomy incision is to be made. After placing device 20 an episiotomy can be performed while the delivery process continues, within opening zone 50 of frame 60. FIG. 1A is a side view of device 20 placed on fourchette 70 during delivery of woman 10 (shown in FIG. 1). Numeral 75 indicates further tissues underlying fourchette 70 that can be incised during performance of episiotomy. The additional tissues may include the fascia and muscles of the perineal body (second degree). Episiotomy can be performed either immediately after placing device 20 on fourchette 70, at a later stage of the deliver, or not at all in accordance with the situation assessment during the delivery made by the midwife or physician during the delivery process. The present location of device 20 shown in FIGS. 1, 1A and 2 illustrates performance of episiotomy according to the mediolateral type mentioned above. However, one skilled in the art can appreciate that device 20 can be placed to perform a midline episiotomy as well. Thus, device 20 can be placed on the vagina wall and partially on the perineum substantially aligning with the anus. Device 20 located on fourchette is substantially fixed on tissue 70 by friction of frame 60 bottom surface, facing tissue, with fourchette 70. As depicted below other means can be provided for substantially fixing a device disclosed in the present invention to a tissue.

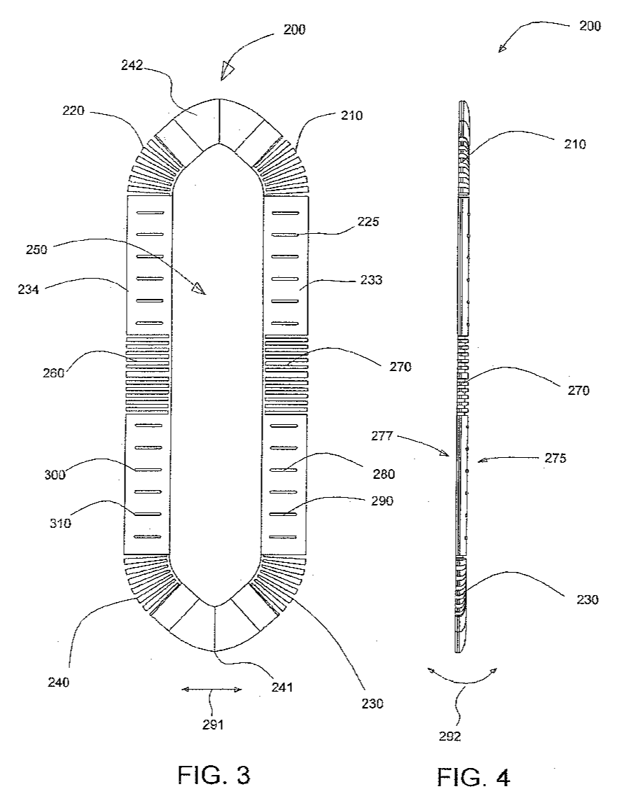

FIG. 2 is a schematic illustration of device 20 after performance of episiotomy incision 100 has already been made to assist with the delivery process. Device 20 comprises opposing side s 110, 120, and opposing ends 130 140. In accordance with the embodiment shown here, sides 110, 120 and ends 130, 140 are integrally connected to each other by flexible joints, forming a flexible polygonal frame 60. Frame 60 is deformable in both in plane and out of plane directions so as to allow folding the frame to conform to the forchette as well as to increase the distance between side sections 110 and 120 while keeping the frame around fourchette wall 70. Thus, providing reinforcement of cut edges and facilitating subsequent cosmetic efficient and fast closing of cut tissue. A detailed view of frame 60 in its rest position, i.e., where no external forces are exerted on the frame, is depicted in FIGS. 3 and 4. FIG. 2 also provides a view of incision 100 performed within frame 60. Undergoing episiotomy, an incision 100 of tissue 70 is made within opening 50 of frame 60. Incision 100 comprises, a longitudinal section bordered by sides 170, 180, and ends 150, 160. Frame 60 further comprises flexible joints sections 170 through 178. According to the present embodiment incision 100 is made after device 20 is placed on fourchette 70. Incision 100 is performed in a manner that its ends 150, 160, are substantially opposing each other, and are aligned with ends 140, 130 of frame 60, respectively. Sides 110, 120, of frame 60 are substantially parallely and adjacent to sides 170, 180, of the incision 100. Though the present embodiment presents that the device is located on a tissue before performing an incision (e.g episiotomy), other embodiments can provide the use of a device as disclosed in this invention, wherein the device is placed on a tissue outlining an incision after said incision is made. Frame 60 outlines the surrounding tissue area of an incision and hinders the capability of an incision to expand beyond sides 110, 120, and ends 130, 140 of the frame. Arrow 90 and arrow 155 indicate the direction of lateral forces and longitudinal forces, respectively, applied on incision 100 during delivery. Both lateral forces and longitudinal forces are due subject to the delivery process that applies substantial forces by the emerging fetus. Thus said lateral forces apply opposing forces on sides 170, 180 that widens the opening of incision 100. Longitudinal forces apply opposing force on ends 150, 160 that can expand the tear of incision 100. Furthermore, said lateral and longitudinal forces applying on incisions made deliberately by a surgeon, or alternatively, incisions created subject to the lateral forces tend to expend the size and dimensions of said incisions, thus widening a longitudinal cut, deepening the incision (cutting and damaging deeper tissues), and lengthening the cut, perpendicularly to the radial forces. Nevertheless, due to the flexibility of frame 60 and flexible sections 171 through 178, sides 110, 120 advance in the same direction as sides 170, 180, of incision 100, respectively. The advancement of sides 110, 120 is limited to the flexibility of frame 60 and flexible sections 171 through 176. Similarly, the position of ends 150, 160 of incision 100 is limited to the flexibility of frame 60 and its flexible sections 171-178. The sections 171-178. The location of device 20 vies-a-vise to incision 100 and its location on fourchette 70 affect the capability of incision 100 to expand during delivery. Thus, frame 60 juxtaposed to fourchette 70 and encircling incision 100, restricts the expansion of incision 100 by adding significant reinforcement to tissue encircling incision 100. Frame 60 limits the capability of incision 100 from expanding beyond to sides 110, 120, and ends 130, 140, of the frame. The location of device 20 is determined substantially by placing device 20 on the desired tissue (e.g. fourchette 70), and the contact of the bottom surface of frame 60 and fourchette 70. The contact of bottom surface of frame 60 with fourchette 70 consequently attaches device 20 to fourchette 70 due to the material fabricating frame 60. Device 20 is preferably made of polystyrene polymer. Other semi flexible materials may be used such as rubber and the like. Other materials approved for medical use can also be used. In some embodiments of the invention biodegradable materials can also be sued. Top surface generally facing away from female body is preferably smooth while undersurface or bottom surface (i.e. generally facing female body) of body 60 maybe rugged and covered with a bonding material sauch as water favorable glue that attaches said undersurface of device 60 to the mucosa and skin of female.

FIGS. 3 and 4 are a top and side view, respectively, of device 200 which is another examplary embodiment of the present invention. FIGS. 3 and 4 show device 200 in its rest planar shape, substantially unbent and not stretched. Device 200 comprises generally a rectangle frame 225 that has side sections 233, 234, and end sections 241, 242 connected by flexible joints 210, 220, 230, 240. Side sections 233, 234 further comprise flexible mid joints 270, 260, respectively. The flexible joints 210, 220, 230, 240, 270, 260, determine the flexibility of frame 225. Thus, side sections 233, 234 can advance in opposite sides as shown in arrow 291. Additionally, side sections 233, 234 can be bent over in direction shown in arrow 292. Device 200 used for assisting an episiotomy is placed on a tissue (e.g. fourchette) encircling an incision having a longitudinal part with two ends, as discussed above, is performed. Device 200 is having a bottom surface that is placed on the tissue, and an upper surface 275 that is opposing the bottom surface 277. Device 200 is located in fashion that the incision's (not shown) ends align both end sections 241, 242, and longitudinal part of the tissue is substantially parallel aligning side sections 233, 234. Frame 225 in FIGS. 3 and 4 is a substantially rectangle planar shape, however placing device 200 juxtaposed to or on a tissue as shown in FIGS. 1, 1A, 2 may require bending of the device 200, as shown in FIG. 1A. Additionally, device 200 substantially encircling and outlining an incision that is under lateral forces as shown in FIG. 2, requires flexibility of side sections 233, 234. One skilled in the art can appreciate that flexible joint sections 210, 220, 230, 240, 270, 260, provide device 200 also with a rotational bending capability that can be required when placing the device on a tissue that is not a flat surface. Flexible joints 210, 220, 230, 240, 270, 260 provide device 200 with stretching abilities enabling the deforming of frame 225. However, the stretching capabilities of flexible joints 210, 220, 230, 240, 270, 260 is limited due is to the fabricating material constrains. Hence, the stretching of side sections 233, 234 advancing in opposite directions, as shown in arrow 291, will end as the stretching sections will meet their stretching limit. Thus, reaching the stretching limits of flexible joint sections 210, 220, 230, 240, 270, 260 will hinder or slow down the expansion of an incision encircled by frame 225 in the direction of side sections 233, 234. The flexibility of device 200 subjected to the movements of ends 241, 242 in opposing direction, either towards each other, or away from each other, is limited. Thus, device 200 limits an expansion of an incision (not shown) encircled by frame 225 beyond end sections 241, 242. Flexible joint sections 210, 220, 230, 240, 260, 270 are areas of frame 225 can be comprised of stretchable polymer or folds of material that comprise the other sections of frame 225. Thus, said flexible sections allow relative movement of parts 223, 224, 241, 242 around said flexible sections.

Device 200 comprises further visual indicators 280, 290, 300, 310. Indicators 280, 290, 300, 310 are marking lines located on the upper surface 275 of device 200. Indicators 280, 290 on side 233, are opposing indicators 300, 310 on side 234, respectively. Due to fact that device 200 is to be placed on a tissue outlining an incision (not shown), indicators 280, 290, 300, 310 indicate for a surgeon locations where to suture the tissue when required (e.g. after delivery is ended). Indicators 280, 290, 300, 310 provide a surgeon a substantially precise and a quick and easy way to locate the vicinity of sides of the incision before suturing, such that anatomy of cut is restored in optimum fashion. One skilled in the art can appreciate that other embodiments can comprise less, or alternatively, more indicators. Other embodiments can comprise other types of indicators such as indicators comprising any outstanding color, a different texture of material in comparison to the rest of the upper surface of a frame, and the like.

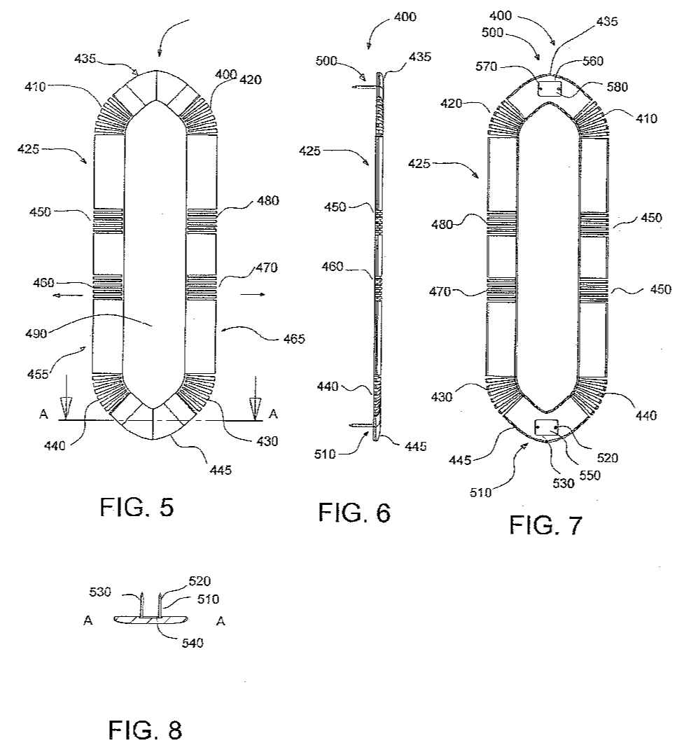

FIG. 5 is a top view of device 400 which is another exemplary embodiment according the present invention. FIGS. 6, 7, are a side and bottom view of device 400, respectively. Device 400 comprises an upper surface viewed in FIG. 5 and a bottom surface viewed in FIG. 7. Device 400 comprises a frame 425 that comprises side sections 455, 465, and end sections, 435, 445. Frame 425, as depicted in previous embodiments, is configured to be located on a tissue, and substantially outlining an incision performed during a surgical procedure (e.g. episiotomy). According to the preferred embodiment of the invention, an incision (not shown) will be initiated by a midwife or surgeon within on a tissue outlined by frame 425 and opening 490. Preferably, the incision is made that the longitudinal section is initially parallel aligning to sides 455, 465, and incision's ends align with ends 435, 445. Similarly to the depicted embodiments sides 455, 465, and opposing ends 435, 445, are integrally connected. Sides 455, 465, comprises opposing flexible joint sections 450, 460, and 470, 480, respectively. Thus, each of the sides comprises two flexible sections. One skilled in the art can appreciate that other embodiments can provide that sides of device can have a plurality of flexible sections, or alternatively, the sides of a frame can be all or substantially all flexible. Furthermore, other embodiments can have unidentical opposing flexible sections. Alternatively, a device can have no visible flexible sections at all. The side view of device 400 in FIG. 6 shows attaching means 500, 510. Attaching means 500, 510 are located in the bottom surface of frame 425 within ends 435, 445, respectively. Attaching means 500, 510 are preferably staple pin like members that are used to attach device 400 to the tissue encircling the incision. FIG. 8 is a cross section of end 435 of device 500 at line AA indicated in FIG. 5. Pins 500, 510 comprise each two penetrating pointed rods 520, 530, and 570, 580, respectively. The length of rods 520, 530, 570, 580, can range preferably between about 0.1-2 centimeters. Size may be longer for devices for use with non-human patients. Rods 520, 530 are affixed to base 540, and rods 570, 580 are affixed to base 560. Device 400 can be located on a fourchette of female delivering. Attaching device 400 to a fourchette using attaching means assures that device 400 will be substantially affixed to its initial location, thus encircling the incision. Furthermore, affixing device 400 with attaching means 500, 510 further hinders or slows down the capability of the incision to expand beyond attaching means. Thus, the presence of pins 500, 510 within the encircling tissue at ends 435, 445 aligned with the longitudinal section of the incision further prevents the incision to expand in the longitudinal direction. Further attaching means can be provided at other locations alone frame 425 undersurface (i.e. surface generally facing female body), such as but not limited to, sections 457, 467 undersurface, each part of sections 455, 465, respectively. These attaching means may prevent or slow down spreading of an incision in lateral direction. Other attaching means may be placed along other placed of undersurface of frame 425. As shown in FIGS. 5, 6, 8, bases 540, 560 are not projecting in the upper surface of frame 425. Thus, device 400 does not present an obstacle for the fetus within the delivery process. One skilled in the art can appreciate that according to other embodiments a base of an attaching means can be within width of frame 425 and not visible. Other embodiments can be provided attaching means that is an adhesive, such as a bio-compatible clue and the like, or alternatively, a combination of pins and adhesive. Furthermore, attaching means in other embodiments can be located only on one end of a frame, on both ends and on one or more sides of a frame. Attaching means can be located all around a bottom surface of a device according to the present invention.

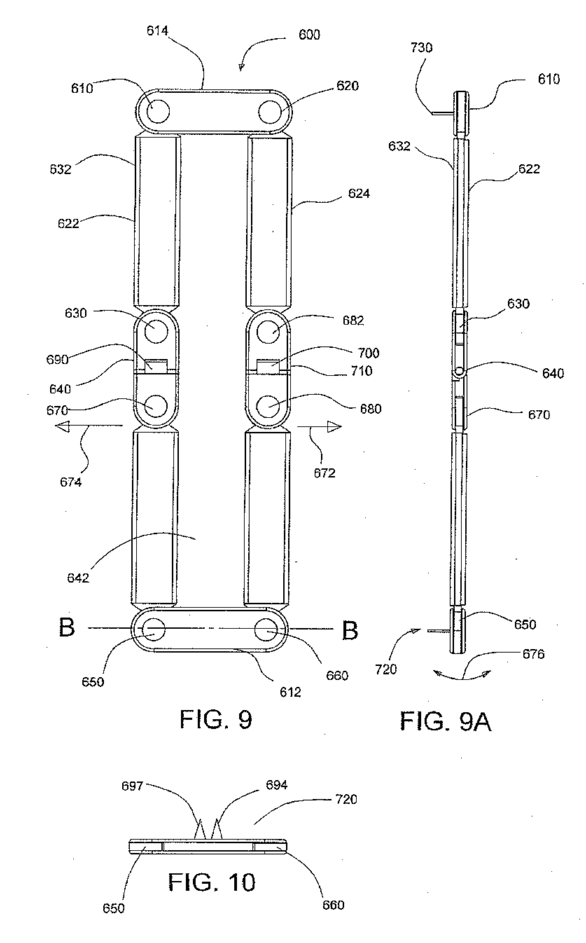

FIGS. 9 and 9A are top and side views of a further exemplary embodiment of the present invention. Device 600 comprises generally a substantially rectangle frame 632 which comprises opposing sides 622, 624, and opposing ends 612, 614, that are integrally connected. As depicted above also device 600 is an embodiment for using within a surgical procedure involving an incision. Device 600 can be placed on a tissue encircling the incision as depicted above. Side 622 comprises hinges 630, 640, 670, and side 624 comprises hinges 680, 682, 710. Ends 612, 614 comprises hinges 650, 660, and 610, 620, respectively. Hinges 630, 670, 680, 682, provide frame 632 with the flexibility of sides 622, 624 to move in the direction of arrows 674, 672. Sides 622, 624, comprise further joints 690, 700. Joint 690 is coupled to hinges 630, 670, together with hinge 640, and joint 700 is coupled to hinges 680, 682, with hinge 710. Thus, hinges 640, 710 together with joints 690, 700, respectively, provide flexibility of frame 632 in the direction shown in arrow 676. Device 600 is different from the previous embodiments by comprising hinges instead of flexible sections that provide the flexible capability enabling the deformation of frame 632. Similar to the deformation limits discussed above regarding previous embodiments, it is evident that the hinges within device 700 provide a limit to the deformation of frame 632. Hinges of frame 632 may be covered by protecting material associated (e.g. attached) with sides 614, 612, 622, 624, such as to protect underlying tissue and overlying parts from their movement during the labor process. FIG. 10 is a cross section view of end 612 at line BB of device 600. Device 600 comprises further attaching means 720, 730 that are positioned at ends 612, 614, respectively. Attaching means 720, 730, are another embodiment of attaching means. Attaching means 720, 730, are staple like pins that comprise triangle shape pins that attach frame 632 to the tissue encircling the incision. Thus, pin 720 comprises two triangle shape pointed rods 697, 694. Rods 697, 694, can be connected with a base (not shown) positioned within end 612. The triangle shape of rods 692, 694, provides a further effective measure to prevent expansion of the incision in the direction of ends 612, 614 of frame 632. As depicted within previous embodiments said attaching means can be multiplied along frame 632.

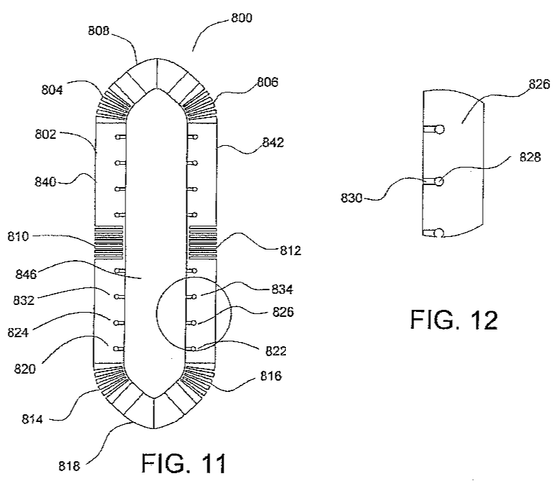

FIG. 11 is a top view of a further embodiment according to the present invention. Device 800 is substantially the same as device 200 depicted in view of FIGS. 3 and 4. Similarly, device 800 is configured to be located on a tissue encircling a tissue. However, device 800 comprises further repair assisting means 820, 822, 824, 826, 832, 834. Similarly to device 200 also device 800 comprises frame 802 that comprises sides 840, 842, and ends 808, 818. Frame 802 comprises also flexible sections 804, 806, 810, 812, 814, 816. Repair assisting means 820, 824, 832, and 822, 826, 834, are opposing each other, and are positioned on sides 840, 842, respectively. FIG. 12 provides a blow up of a single assisting repair means 826. Repair means 826 provides an aperture 828 and an extending breakable material 830. Aperture 828 provides a location for suturing the incision. Breakable extension 830 provides the possibility to easily tear off frame 802 from the tissue after suturing the incision was performed. According to other embodiments there is no material but a slit between opening 846 and apertures within sides of frame.

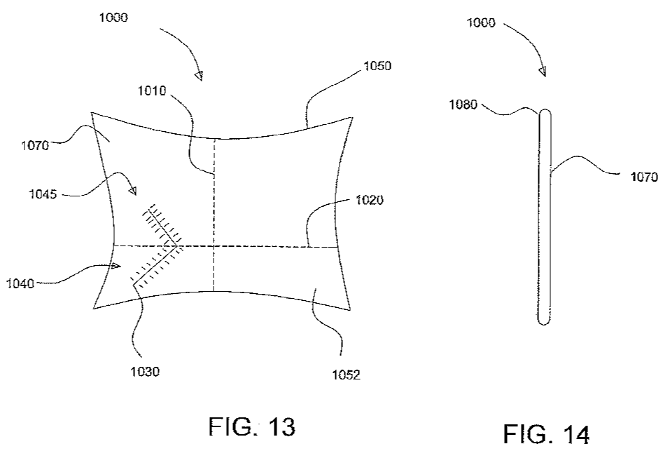

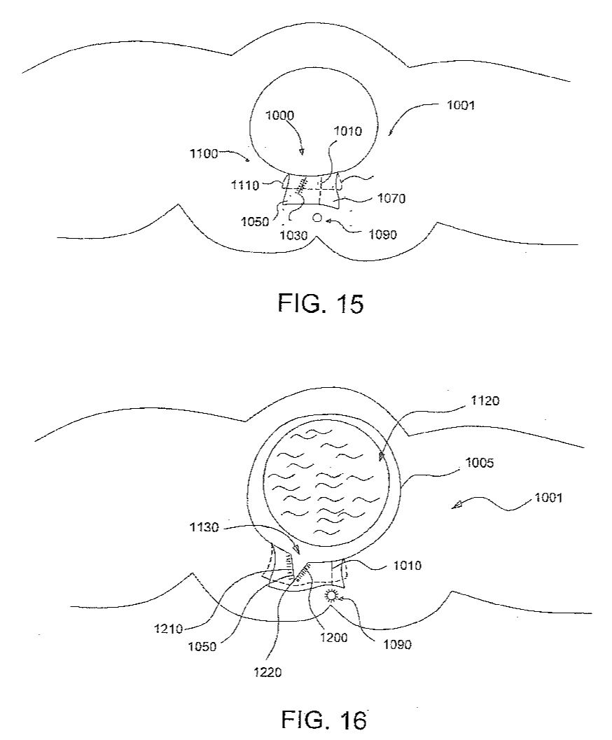

Another aspect of the present invention is disclosed in view of FIGS. 13, 14, 15, 16 and 17. Device 1000 shown in FIG. 13 is in its rest planar shape, substantially unbent and not stretched. Device 1000 is a generally a rectangle sheet that comprises an upper surface 1070 and an undersurface 1080. Upper surface 1070 generally faces away from female body (not shown) and undersurface 1080 generally faces the female body. As shown in FIG. 14 device 1000 is a planar shape in its rest position. Device 1000 is positioned on a tissue of a female before performing an incision (e.g. episiotomy). Thus device 1000 when placed on fourchette outlines a tissue (not shown) underlying the device (as shown in FIG. 15). Device 1000 further comprises a folding line 1020, longitudinal middle line 1010, and cutting line zone 1045. Cutting line zone comprises a cutting line 1030 and visual indicators 1040. In accordance to the present embodiment of the invention device 1000 can be positioned on a fourchette 1100 of a female 1001 as shown in FIGS. 15, 16, 17. Accordingly, folding line 1020 indicates the manner that device 1000 is positioned on fourchette 1100. Section 1052 is positioned generally on a first surface of fourchette 1100 (e.g. on the surface generally facing away from woman 1001), and surface 1110 is generally positioned on a second surface of fourchette 1100 (not shown) that is indicated in FIG. 15 with broken lines 1112 in order to show the folding of device 1000 at said folding line 1020. Middle line 1010 indicates to a person (not shown) wishing to position device 1000 on fourchette of female 1001 where to place the device. One exemplary use of middle line 1010 can be when an episiotomy will be used in a semi-midline fashion. Thus, device 1000 is placed on a fourchette in a manner that middle line 1010 is alligned with anus 1090 as shown in FIGS. 15, 16. Device 1000 is generally positioned on a fourchette of delivering female 1001 during the delivery process, and before fetus 1120 (shown in FIG. 16) emerges through the birth canal. Due to the fact that device 1000 outlines an underlying tissue, locating device 1000 on fourchette 1100 reinforces and strengthens the fourchette's tissue beneath the device and prevents and reduces the tearing of fourchette during delivery. As depicted above, during delivery there are substantial leteral and longitudinal forces that apply on a fouchette of a female undergoing delivery. These forces can cause occurrence of tears of tissue at fourcette, alternatively, existing tears can rapidly expand laterally and longitudinally. Furthermore, attaching device 1000 to fourchette using one or more of the attaching means depicted above (e.g. water affiliated glue) prevents occurrence of new tears and slows down expansion of existing tears beyond the tissue outlined by the device.

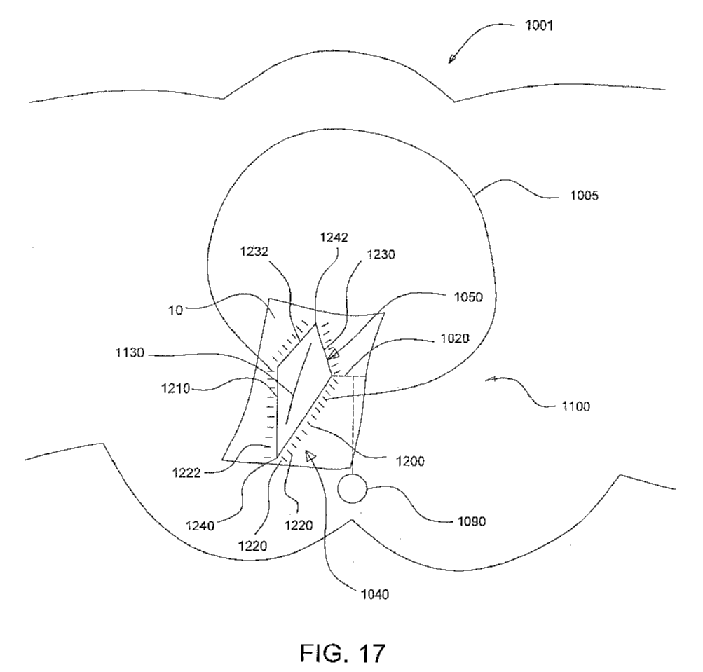

FIGS. 16 and 17 show device 1000 wherein cutting line 1030 is used for cutting an incision 1130 and performing episiotomy. Accordingly, device 1000 is located on fourchette 1100 of female 1001 before fetus 1120 emerges. Device 1000 is placed on fourchette 1100 according to the guidance of longitudinal middle line 1010 and folding line 1020. Thus, longitudinal middle line 1010 is aligned with anus 1090 and folding line 1020 undersurface (not shown) is positioned on the fold of fourchette 1100. After device 1000 is folded and bent on fourchette 1100, cutting line is used for indicating the cutting with surgery scissors (not shown) for performing incision 1130. Incision 1130 aids the delivery of fetus 1120 by enlarging circumference of the outlet 1005 of the birth channel. After performance of incision 1130 a rhombus shape frame 1050 is received. Frame 1050 comprises sides 1200, 1210, 1230, 1232, and ends 1240, 1242. Device 1000 can be attached to the underlying tissue by any of the attaching means depicted above. Due to the presence of device 1000 and frame 1050 the expansion of incision 1130 is prevented or slowed down. Thus, device 1000 reinforces tissue underlying the device and hinders or slows down expansion of incision 1130 in direction of ends 1240, 1242, and sides 1200, 1210, 1230, 1232. Indicators 1040 are positioned along the bordering end of frame 1050 with incision 1030. Indicator pairs 1220, 1222, respectively, are exemplary indicators that assist to suture incision 1030 after fetus 1120 emerged from outlet 1005. The indicator pairs indicate the relative position of sides 1200, 1210 to each other before cutting incision 1130 open. Suturing the tissue (not shown) underlying frame 1050 by using indicator pairs 1220, 1222. Indicator lines are marked all along rhombus shape frame 1050 also lining sides of rhombus 1232, 1230. Said indicators provide assistance for accurate suturing of the tissue to reconstruct the pre incision 1030 stage=

According to other embodiments of the invention a device and frame can be transparent and enabling to show a midwife or physician of the tissue underlying said device.

It will be appreciated by persons skilled in the art that the present invention is not limited to what has been particularly shown and described hereinabove. Rather the scope of the present invention is defined only by the claims, which follow.

1. A device for protecting soft tissue and for assisting in the performance of surgical procedure involving incision of tissue, the incision having a longitude section and two ends, the device comprising a flexible sheet configured to be placed on the tissue, wherein the sheet outlines the tissue.

2. A device according to claim 1 wherein the device is flexible.

3. A device according to claim 1 wherein the device further comprises a frame.

4. A device according to claim 3 wherein the frame outlines the tissue.

5. A device according to claim 1, wherein the frame flexibility enables deformation of the device relative to the tissue.

6. A device according to claim 1, wherein the frame comprises,
at least two opposing sides; and
at least one end;
wherein the sides and the at least one end are integrally connected.

7. A device according to claim 1, wherein each end of the frame is configures to be placed adjacent to one of the ends of the incision.

8. A device according to claim 6, wherein the end of the frame is configured to placed adjacent to the end of the incision.

9. A device according to claim 8, wherein the end of the frame is further substantially aligning to the longitude section of the incision.

10. A device according to claim 1, wherein the frame comprises at least one flexible section enabling the deformation of the frame.

11. A device according to claim 10, wherein the flexible section is integrally connected with the frame.

12. A device according to claim 10, wherein the flexible section comprises at least one hinge.

13. A device according to claim 1, wherein the device is attachable to the tissue.

14. The device according to claim 13, wherein the frame comprises a top surface and bottom surface, and wherein at least one section of the bottom surface comprises attaching means for attaching the device.

15. A device according to claim 14, wherein the attaching means comprise an adhesive.

16. A device according to claim 15, wherein the adhesive is bio-compatible glue.

17. A device according to claim 14, wherein the attaching means comprise at least one bio-compatible pin.

18. A device according to claim 17, the pin is a stapler pin like.

19. A device according to claim 1 wherein at least one section of the frame comprises repair assisting means enabling to assist repair of the incision.

20. A device according to claim 6, wherein each of the sides of the frame is configures to be placed on opposing sides of the incision.

21. A device according to claim 20, wherein at least one section of the sides of the frame comprises repair assisting means for assisting repair of the incision.

22. A device according to claim 21, further comprising at least two opposing sections of the frame with repair assisting means wherein each of the opposing sections comprises at least one visible indicator for indicating a location where to suture the tissue.

23. A device according to claim 21, wherein the assisting means comprise at least one pair of apertures placed on substantially opposing sides, enabling to suture the tissue and repair the incision.

24. A device according to claim 23, wherein at least one portion of the section of the frame comprising an aperture is breakable.

25. A device according to claim 1, wherein said surgical procedure is episiotomy.

26. A device according to claim 1, wherein said surgical procedure is performed on people.

27. A method for protecting soft tissue and for assisting in the performance of surgical procedure involving an incision of a tissue, the method comprising placing a device comprising a deformable frame on a location outlining the tissue.

28. A method according to claim 27, wherein the frame further comprises, at least two opposing sides, and at least one end.

29. A method according to claim 28 further comprising the step of substantially fixing the at least one end of the frame adjacent to the incision.

30. A method according to claim 28 further comprising the step of substantially fixing an at least one side of the frame adjacent to the incision.

31. A method according to claim 28 wherein the frame comprises attaching means for attaching the frame, further comprising the step of attaching the frame to the tissue or the tissue's vicinity.

32. The method according to claim 27 used for performing episiotomy.

33. The method according to claim 27 used for humans or animals.
